(19) United States
(12) Patent Application Publication
(10) Pub. No.: US 2008/0011304 A1
(43) Pub. Date: Jan. 17, 2008

(54) Endotracheal tube with suction attachment

(76) Inventor: Fermin V. G. Stewart, Duluth, GA (US)

Correspondence Address: VORYS SATER SEYMOUR PEASE, 1828 L STREET NW, ELEVENTH FLOOR, WASHINGTON, DC, 20036, US

(21) Appl. No.: 11/797,653

(22) Filed: May 4, 2007

Related U.S. Application Data
(60) Provisional application No. 60/797,370, filed on May 4, 2006.

(51) Int. Cl. A61M 16/04

(52) U.S. Cl. 128/207.15

(57) ABSTRACT

An improved endotracheal tube having a suction sleeve circumferentially thereabout with a plurality of suction holes or ports therein. The suction sleeve is spaced a distance above an inflatable balloon or cuff so that the vocal cords of a patient may rest between the sleeve and the cuff, and the suction sleeve, sealed at its top and bottom to the endotracheal tube, has a plurality of circumferentially and longitudinally-spaced suction holes therethrough for suctioning the pools of secretion that form in the hypopharynx and lower oropharynx of an intubated patient. Tubing is also provided for connecting a suction chamber between the suction sleeve and the main lumen of the endotracheal tube to a well-known low intermittent suction (“LIS”) device.

### CROSS-REFERENCE TO RELATED APPLICATION

This application claims priority from U.S. Provisional Application No. 60/797,370, filed on May 4, 2006, which is incorporated herein by reference.

### FIELD OF THE INVENTION

The present invention relates, in general, to medical devices, and in particular, to endotracheal tubes inserted through the nose or mouth of a patient with impaired respiratory function.

### BACKGROUND OF THE INVENTION

Endotracheal tubes are well-known for inserting through the mouth or nose and down the throat of a patient who has lost control of his or her breathing due to anesthesia or coma. Such endotracheal tubes are characterized by a main lumen having proximal and distal ends, and an inflatable cuff or balloon, secured around the main lumen adjacent the distal end, for inflation within the trachea of the patient. The balloon is inflated within the trachea of the intubated patient in an attempt to seal the trachea and thereby block the flow of secretions that form in the mouth, oropharynx, and hypopharynx from passing into the lungs during the intubation period in which the epiglottis, which normally seals the trachea during swallowing, is held open by the inserted endotracheal tube. Typically, as much as a liter to a liter and a half of secretions flow into the hypopharynx during a day. However, the seal formed by the balloon is imperfect and, as the trachea tissues expand around the inflated balloon, the secretions pass down the trachea past the balloon and into the lungs, causing medical complications. Additionally, because the intubated patient has lost all ability to swallow, secretions pool and accumulate in the hypopharynx and lower oropharynx, where bacteria then grows.

Prior art devices have attempted to suction the flowing secretions through suction ports at or near the inflated balloon, but, by the time the secretions reach such the regions of the trachea near the balloon, it is virtually impossible to prevent the secretions from flowing past the poorly sealing balloon and into the lungs.

It is therefore desirable to have an improved endotracheal tube that removes the pools of secretions that heretofore have formed in the hypopharynx and lower oropharyngeal regions of an intubated patient, without damaging the vocal cords which are situated just above the inflated cuff balloon of the endotracheal tube.

U.S. Pat. No. 4,305,392 to Chester, issued Dec. 15, 1981, discloses an endotracheal tube of the inflatable cuff type, having a suction chamber adjacent the upper side of the cuff. The suction chamber is in the shape of a bulge having four ports equally spaced about the periphery of the bulge and facing upwardly. Suction applied to the chamber will extract fluids from the trachea above the cuff without invaginating the tracheal mucosa. Medicinal fluids may also be introduced into the trachea via the suction chamber and ports.

U.S. Pat. No. 4,584,998 to McGrail, issued Apr. 29, 1986, discloses a multi-purpose tracheal tube for use with high frequency ventilation. The tube is an endotracheal tube including up to three lumens, in addition to the primary lumen, which serve various functions to provide versatility in the treatment of patients. In cuffed tubes one of the lumens is used for inflating the cuff once the tube has been placed in the desired position in the trachea of the patient. Another lumen, referred to as the “insufflation lumen”, is used to deliver oxygen or other gases by constant insufflation, intermittent jet ventilation or high frequency ventilation. The third lumen, when incorporated, is employed for monitoring and irrigation. The distal opening of the irrigation or monitoring lumen is located just inside the distal tip of the tube while the insufflation lumen opening is located rearwardly toward the proximal end of the tube relative to the irrigation or monitoring lumen opening.

U.S. Pat. No. 4,607,635, to Heyden, issued Aug. 26, 1986, discloses an endotracheal tube that is adapted to incorporate an elongated passage along its length. Ports are located along the elongated passage and arranged to provide for removal of secretions that accumulate outside the endotracheal tube and between the endotracheal tube and a substantial length of the intubated pathway wall when the endotracheal tube is in place. The positioning of ports is such that direct contact of the port openings with the mucosa is avoided, thereby minimizing blockage of these openings. The elongated passage provides a shield for a suction catheter which is insertable into the elongated passage and used to transport the secretions out of the intubated pathway. The suction catheter is easily removed to allow for cleaning or replacement.

U.S. Pat. No. 4,632,108, to Geil, issued Dec. 30, 1986, discloses a flexible tubular assembly that has a distal end disposed within the trachea of a patient and a proximal end outside the body of the patient. An inflatable balloon carried on the distal end of tubular assembly can be inflated into sealing contact with the trachea. A first conduit in the tubular assembly conveys anesthesia and ventilation gases through the assembly and a second conduit is used to inflate the balloon. The flexible tubular assembly includes a polymeric matrix having a reflective filler embedded therein. The filler includes finely divided particles having a metallic surface coating which is reflective to infrared laser radiation. The tubular assembly also includes a smoke removal lumen with an opening proximal the balloon to remove smoke generated during laser surgery.

U.S. Pat. No. 4,762,125 to Leiman et al., issued Aug. 9, 1988, discloses a balloon-tipped suction catheter, for extirpating and aspirating tracheobronchial secretions from the trachea, endotracheal tube or tracheostomy tube and a method for using such catheter. The catheter preferably embodies an elongated tube with a cannula extending along the elongated tube. The distal end of the cannula communicates with an expandable membrane or balloon which is attached either to the walls of the distal end of the elongated tube or directly to the distal end of the cannula. Proximal to the expandable membrane are apertures which provide fluid communication between the exterior and interior of the elongated tube. A control port is provided at the proximal end of the catheter to control the transmission of suction through the catheter. In use, the catheter of this invention is inserted into an endotracheal tube, for example, to remove secretions. Positive end expiratory pressure (“PEEP”) can be maintained by inserting the catheter through a “Y” connector assembly with a self-sealing diaphragm assembly. After the desired location is reached, the expandable membrane is inflated until it seals against the walls of the passageway. The control port is left uncovered until the membrane is inflated. The catheter is withdrawn and secretions are removed by the squeegee action of the balloon wall against the passageway wall and by aspiration.

U.S. Pat. No. 4,840,173 to Porter, III, issued Jun. 20, 1989, discloses an endotracheal tube having dual passages provided by the merging of a ventilation tube and a suction tube. The ventilation tube is adapted to project into a patient's trachea to the position of the carina anterior. An inflatable cuff surrounds the ventilation tube and prevents passage of fluids to and from the lungs. The suction passage terminates at the cuff with openings into the suction passage for suctioning secretions that pool around the cuff. The tubes as merged enable entry of the dual passages into the trachea past the vocal cords. As protruded from the patient's mouth, the tubes are separated to be connected to the respective ventilation machine and secretion suction machine.

U.S. Pat. No. 5,067,497 to Groear et al., issued Nov. 26, 1991, discloses a tubular assembly which can be intubated within a body passageway that comprises a main tube body, which permits the passage of fluid in either direction (such as air in the case of an endotracheal tube), an inflatable cuff near the distal end of the main tube body for locating and securing the tubular assembly within a body passageway as well as for sealing the space above the cuff between the main tube body and the body passageway from the body passageway below the cuff, and a suction tube that runs along the main tube body from the proximal end that remains external of the patient during intubation and a point adjacent the inflatable cuff. The suction tube is fixed with the main tube except for an adjustable portion of the suction tube nearest the inflatable cuff that includes the suction tube opening. The adjustment portion of the suction tube is free to move relative to the main tube body such that it is selectively radially adjustable in position to or from the main tube body. Moreover, the adjustable portion of the suction tube is movable by an adjustment balloon positioned between the adjustable portion of the suction tube and the main tube body. Furthermore, the adjustment balloon is selectively inflated or deflated for radially moving the adjustable portion between an innermost position in close proximity to the outer surface of the main tube body, and an outermost position wherein the adjustable portion and suction tube opening are in close proximity to the wall of the body passageway.

U.S. Pat. No. 5,143,062 to Peckham, issued Sep. 1, 1992 an endotracheal tube used for mechanical ventilation of a hospital patient, wherein the endotracheal tube is useful in evacuating contaminated secretions that pool within the trachea above an inflatable cuff associated with the endotracheal tube. The endotrocheal tube of the present invention comprises a double lumen through which air may be circulated, thus creating an indirect gentle suction through a suction eye communicating with the distal ends of the lumens, and located at a position proximal to the inflation cuff. This gentle indirect suction reduces the risk of damage to the tracheal mucosa, which often occurs when applying direct suction.

However, none of these references, either singly or in combination, disclose or suggest the present invention.

### SUMMARY OF THE INVENTION

The present invention is an improved endotracheal tube having a suction sleeve circumferentially thereabout with a plurality of suction holes or ports therein. The suction sleeve is spaced a distance above the inflatable balloon or cuff that is typically found on endotracheal tubes so that the vocal cords of a patient may rest between the sleeve and the cuff, and the suction sleeve, sealed at its top and bottom to the endotracheal tube, has a plurality of circumferentially and longitudinally-spaced suction holes therethrough for suctioning the pools of secretion that form in the hypopharynx and lower oropharynx of an intubated patient. A tube is also provided for connecting the suction sleeve to a well-known low intermittent suction (“LIS”) device.

It is an object of the present invention to prevent the pooling of secretions in the hypopharynx and lower oropharyngeal regions of an intubated patient by suctioning the secretions as they are generated, thereby reducing the medical complications that would otherwise occur due to the growth of bacteria in the secretion pools or due to the passage of the secretions into the lungs.

### BRIEF DESCRIPTION OF THE DRAWINGS

FIG. 1 is a partial sectional view of a patient's throat showing the present invention in place.

FIG. 2 is a partial longitudinal sectional view of the present invention.

FIG. 3 is a partial longitudinal schematic view of the present invention according to a preferred embodiment.

FIG. 4 is an enlarged transverse sectional view of the present invention through the suction sleeve, taken substantially along the line 4-4 shown in FIG. 2.

FIG. 5 is a schematic representation of a hook that may be used with the present invention.

FIG. 6 is a schematic cross section of a patient's head showing the areas targeted by the present invention.

### DESCRIPTION OF THE PREFERRED EMBODIMENT

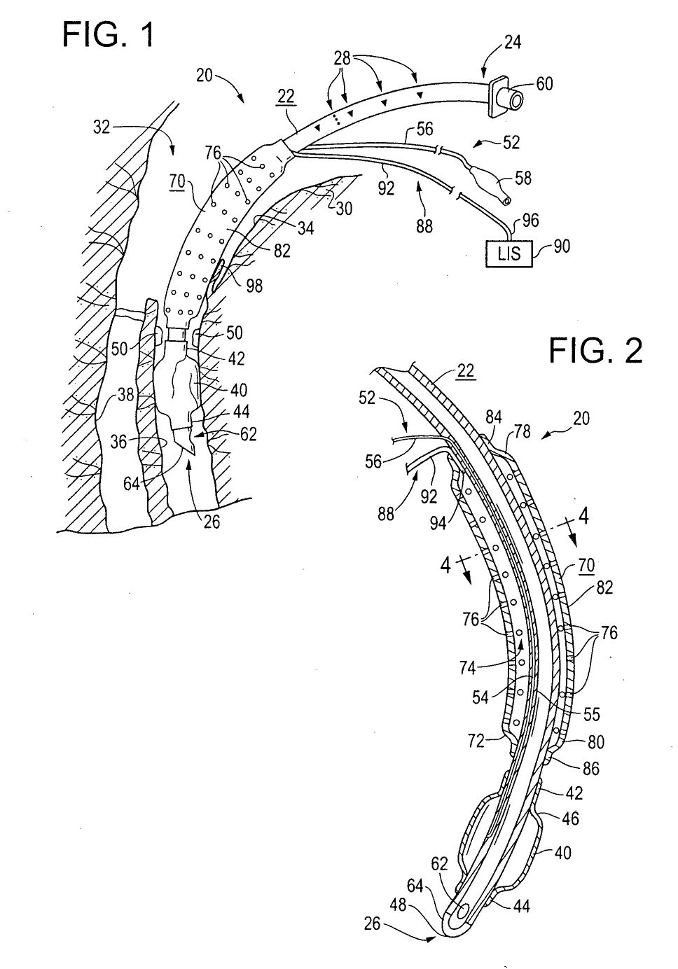

Referring to FIGS. 1-3, endotracheal tube 20 is seen to comprise a main lumen 22 having a proximal end 24 and a distal end 26, with various well-known markings 28 on the exterior of main lumen 22 for measuring the depth of insertion of the lumen into the trachea of a patient. FIG. 1 shows the endotracheal tube 20 in position within an intubated patient's throat, with the tube passing over the rear of the tongue 30 through the lower oropharyngeal regions 32 and along the hypopharynx 34, with the distal end 26 being in the trachea 36 above the corina, not shown. For reference, the esophagus 38 is shown behind the trachea 36, leading to the stomach, not shown.

Just as with prior art endotracheal tubes, the endotracheal tube of the present invention may have an inflatable cuff or balloon 40 circumferentially surrounding main lumen 22 and secured thereto in a manner well-known to those skilled in the art as by sealing the upper and lower reduced-diameter sleeves, 42 and 44, respectively, that extend longitudinally from balloon 40 to the body of main lumen 22 as shown. Typically, the upper portion 46 of balloon 40 is located a distance of five to six centimeters from the tip 48 of main lumen 22, and balloon 40 is typically 3.0 to 3.5 centimeters in length, measured from the intersections of upper and lower sleeves 42 and 44 with balloon 40. It -shall be understood that balloon 40 is placed just below the vocal chords 50 when inflated, and the tip 48 of main lumen 22 must not extend into the corina (not shown) where the main stem bronchi split from the lower end of trachea 36.

Main lumen 22 is preferably constructed in the well-known manner, like prior art endotracheal tubes, of a cylindrical piece of soft flexible plastic tubing so as to not damage the tissues of the intubated patient during insertion and to allow head and neck movement of the patient during intubation. However, the plastic tubing must have sufficient resilience so as not to collapse while the patient is intubated. Inflation means 52 for inflating balloon 40 is also preferably provided comprising a longitudinal passageway 54 formed in the wall 55 of main lumen 22, with passageway 54 being in communication with the interior of balloon 40 as shown in FIG. 2. Inflation means 52 also comprises well-known flexible tubing 56 connected at one end to passageway 54 and connected at the other end to a well-known valve 58 for sealing air within balloon 40 once the balloon is inflated, in a manner well-known to those skilled in the art. It will be understood that valve 58 is in communication with the interior of balloon 40 through the inflation and deflation path created by tubing 56 and passageway 54, allowing balloon 40 to be inflated and deflated in a manner well-known to those skilled in the art by passing air through valve 58.

A connector 60 may be attached to the proximal end 24 of main lumen 22 for connecting lumen 22 to a source of oxygen in a manner well-known to those skilled in the art, and distal end 26 is preferably beveled, as shown, with a hole 62 provided in the wall 55 of main lumen 52 opposite the beveled side 64 of distal end 26. The structure, construction, and use of endotracheal tubes as described above are well-known to those skilled in the art.

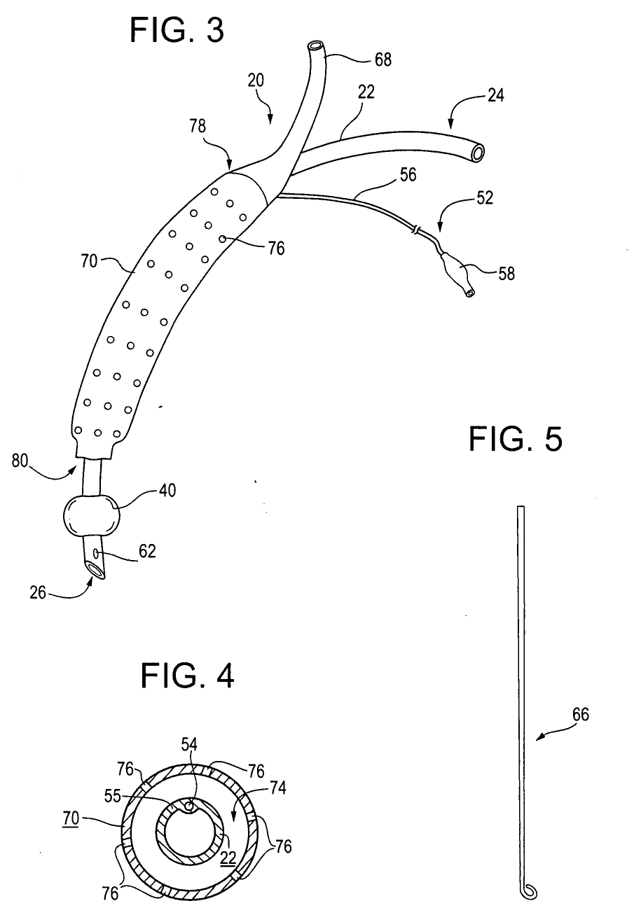

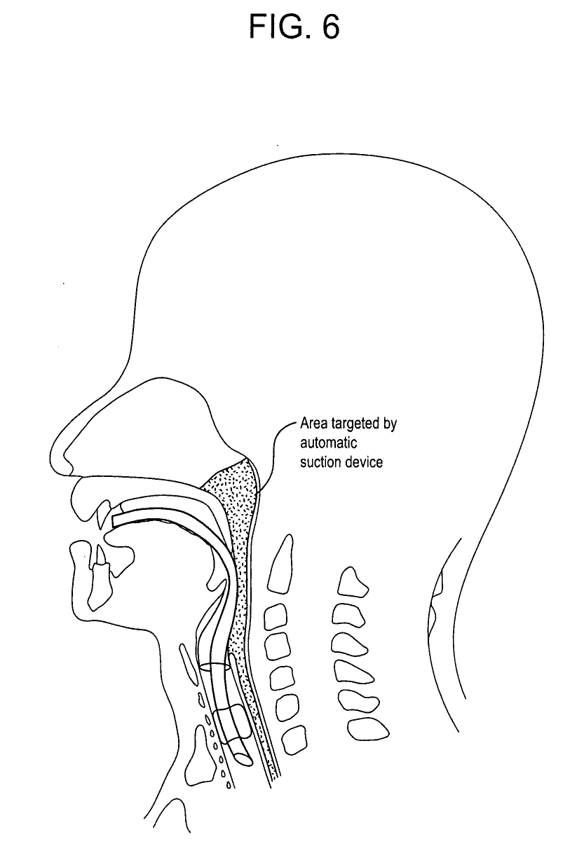

The improvement of the present invention comprises a suction sleeve 70 secured to main lumen 22 between distal and proximal ends 24 and 26, respectively, with suction sleeve 70 being preferably not less than about four centimeters from distal end 26. It shall be understood that the suction sleeve 70 of the present invention may be part of an endotracheal tube that has a balloon 40 as shown in FIG. 1, or may alternatively be a part of an endotracheal tube that does not have a balloon 40. Further, suction sleeve 70 may either be part of the endotracheal tube 20 as shown in FIG. 1, or may be separate from the endotracheal tube 20 as shown in FIG. 3, in which case the suction sleeve 70 is simply slipped over the endotracheal tube 20 as a separate piece. In any case, the distance from the lower expanded portion 72 of suction sleeve 70 to distal end 26 is preferably no less than about four centimeters because the suction sleeve must be located above the vocal chords 50, while distal end 26 must be positioned in the trachea 36 between the vocal chords 50 and the corina (not shown).

As shown in FIGS. 2 and 3, suction sleeve 70 preferably encircles main lumen 22, forming a suction chamber 74 in the region between sleeve 70 and lumen 22, with a plurality of longitudinally-spaced suction ports 76 being through sleeve 70, thereby placing the exterior of sleeve 70 in communication with suction chamber 74. Suction sleeve 70 has a first end 78 and a second end 80, respectively toward proximal and distal ends 24 and 26 of main lumen 22, with first and second ends 78 and 80 being preferably sealed to main lumen 22 so as to make suction ports 76 be the sole communication between suction chamber 74 and the exterior of sleeve 70. Suction sleeve 70 is seen to have a ported portion 82, defined as that portion of sleeve 70 having the plurality of suction ports 76 therethrough, and ported portion 82 preferably has a longitudinal length of not less than about five centimeters so as to extend from just above the vocal chords 50, through the hypopharynx 34, to the lower oropharyngeal regions 32 when the endotracheal tube 20 is placed in a patient's throat, thereby allowing the suctioning of secretions in a manner hereinafter described.

Suction sleeve 70 is preferably located a distance not less than about two centimeters, preferably a distance of two to three centimeters, and preferably not more than about four centimeters, above the upper portion 46 of balloon 40, so as to provide clearance for vocal chords 50 between balloon 40 and suction sleeve 70. It shall be understood that, while first and second ends 78 and 80 of suction sleeve are preferably sealed to the main lumen 22 as shown by reduced-diameter portions 84 and 86, respectively, in a manner well-known to those skilled in the art as by heatingly melting and thereby sealing the plastic of sleeve 70 to main lumen 22, the measurements given herein concerning the preferred distances of sleeve 70 from distal end 26 and balloon 40, and the longitudinal length of ported portion 82, is understood to not include the lengths of reduced-diameter portions 84 and 86 which sealingly contact main lumen 22, or of upper and lower sleeves 42 and 44 of balloon 40.

There is also provided tubing means 88 for placing a source 90 of suctioning vacuum in communication with suction chamber 74. Source 90 is preferably a well-known low-intermittent suction (“LIS”) device, intermittently turning on and off and operating at a vacuum of approximately 20 centimeters of water, in a manner well-known to those skilled in the art. Tubing means 88 preferably includes a length of hollow tubing 92 in communication at one end 94 with suction chamber 74 and in communication at the other end 96 with LIS source 90. The end 94 in communication with suction chamber 74 may be sealed between reduced-diameter portion 84 and main lumen 22 as shown in FIG. 2, or may communicate with suction chamber 74, in a manner similar to that employed by tubing 56 and passageway 54 of inflation means 52, by a second passageway, not shown, longitudinally passing within the wall 55 of lumen 22 and into suction chamber 74, that places tubing 92 in communication with chamber 74, in a manner that will now be understood.

Preferably, ported portion 82 of suction sleeve 70 also includes circumferentially-spaced suction ports 76, with the combined longitudinal and circumferential spacing of the ports from one another serving to populate the surface of ported portion 82 with suction ports 76 as shown so as to have a suction port in the vicinity of any secretion pool that might form in the hypopharynx and lower oropharyngeal regions. It shall be understood to be the intent of the present invention to provide a large suctioning area throughout the region where the secretion pools typically accumulate. Also, rather than requiring the insertion of separate suctioning tubes into the throat of an intubated patient, the suction sleeve 70 of the present invention is correctly positioned for suctioning of the hypopharynx and oropharyngeal regions when the endotracheal tube is in place, thereby ensuring proper suctioning of secretion pools, whether the suction tube 70 is an integral part of the endotracheal tube 20 as shown in FIG. 1 or slips over the endotracheal tube 70 as shown in FIG. 3.

To use the present invention, a doctor or nurse inserts the improved endotracheal tube 20 with suction sleeve 70 through a patient's mouth, past the epiglottis 98, which is held open by endotracheal tube 20 just as with prior art endotracheal tubes, past the vocal chords 50, and into the trachea 36 using markings 28 to determine the depth of insertion. Connector 60 is connected to a source of oxygen, in a manner well-known to those skilled in the art, balloon 40 is inflated by forcing air through valve 58 and then held inflated by sealing valve 58, and LIS 90 is attached to end 96 of tubing 92. LIS 90 can then be activated, in a manner well-known to those skilled in the art, and caused to intermittently suction secretions through ports 76 under the urging of a low vacuum (such as 20 centimeters of water vacuum, for example).

Further, hook 66 is provided as seen in FIG. 5, to grab cuff 68 of the suction device 70. According to a preferred embodiment, hook 66 is preferably made of plastic.

An advantage of the present invention is that, once in place in a patient's throat, a nurse or doctor may now periodically flush or irrigate the patient's throat or nasal passages with water to remove bacteria and thickened secretions, and use the suctioning of the suction sleeve and its many ports to remove the flushing water, now mixed with secretions, from the patient's throat before it passes into the lungs. Such a flushing treatment should help reduce the occurrence of sinusitis to a degree heretofore not possible.

Although the present invention has been described and illustrated with respect to a preferred embodiment and a preferred use therefore, it is not to be so limited since modifications and changes can be made therein which are within the full intended scope of the invention.

What is claimed is:

1. An endotracheal tube for insertion into the trachea, said endotracheal tube comprising:
(a) a main lumen having a proximal end and a distal end;
(b) a suction sleeve secured to said main lumen between said distal and said proximal ends, said sleeve forming a suction chamber between said sleeve and said main lumen and having a plurality of longitudinally-spaced suction ports therethrough; and
(c) tubing means for placing a suctioning source in communication with said suction chamber.

2. The endotracheal tube as recited in claim 1, wherein at least some of said suction ports are circumferentially-spaced from each other.

3. The endotracheal tube as recited in claim 1, wherein said sleeve is secured to said main lumen at a distance not less than about four centimeters from said distal end.

4. The endotracheal tube as recited in claim 3, wherein at least some of said suction ports are circumferentially-spaced from each other.

5. The endotracheal tube as recited in claim 1, wherein said sleeve encircles said main lumen.

6. The endotracheal tube as recited in claim 5, wherein at least some of said suction ports are circumferentially-spaced from each other.

7. The endotracheal tube as recited in claim 1, wherein said sleeve has a first end and a second end, said first and second ends being respectively toward said proximal and distal ends of said endotracheal tube, each of said first and second ends being sealingly attached to said main lumen, and said sleeve has a ported portion defined as that portion of said sleeve having said plurality of suction ports, said ported portion having a longitudinal length of not less than about five centimeters.

8. An endotracheal tube for insertion into the trachea, said endotracheal tube comprising:
(a) a main lumen having a proximal end and a distal end;
(b) an inflatable balloon secured to said main lumen adjacent said distal end;
(c) a suction sleeve secured to said main lumen between said balloon and said proximal end with a distance not less than two centimeters between said inflatable balloon and said sleeve, said sleeve forming a suction chamber between said sleeve and said main lumen and having a plurality of longitudinally-spaced suction ports therethrough; and
(d) tubing means for placing a suctioning source in communication with said suction chamber.

9. The endotracheal tube as recited in claim 8, wherein at least some of said suction ports are also circumferentially-spaced from each other.

10. The endotracheal tube as recited in claim 8, wherein said sleeve encircles said main lumen.

11. The endotracheal tube as recited in claim 10, wherein at least some of said suction ports are circumferentially-spaced from each other.

12. The endotracheal tube as recited in claim 8, wherein said sleeve has a first end and a second end, said first and second ends being respectively toward said proximal and distal ends of said endotracheal tube, each of said first and second ends being sealingly attached to said main lumen, further in which said sleeve has a ported portion defined as that portion of said sleeve having said plurality of suction ports, said ported portion having a longitudinal length of not less than five centimeters.

13. An endotracheal tube for insertion into the trachea, said endotracheal tube comprising:
(a) a main lumen having a proximal end and a distal end;
(b) a suction sleeve placed over said main lumen between said distal and said proximal ends, said sleeve forming a suction chamber between said sleeve and said main lumen and having a plurality of longitudinally-spaced suction ports therethrough; and
(c) tubing means for placing a suctioning source in communication with said suction chamber.
